(19) United States
(12) Patent Application Publication
(10) Pub. No.: US 2021/0068756 A1
(43) Pub. Date: Mar. 11, 2021

(54) CATHETER WITH EXTENSIBLE PRINTED CIRCUIT BOARD

(71) Applicant: Carl Freudenberg KG, Weinheim (DE)

(72) Inventor: Benno SCHMIED, Ludwigshafen (DE)

(21) Appl. No.: 17/012,078

(22) Filed: Sep. 4, 2020

(30) Foreign Application Priority Data
Sep. 10, 2019 (EP) 19 196 488.1

(51) Int. Cl. A61B 5/00; A61B 18/14

(52) U.S. Cl. CPC A61B 5/6852; A61B 2018/00577; A61B 18/1492

(57) ABSTRACT

A catheter includes: an elongate, flexible tube comprising at least one electrode for carrying out a diagnosis or therapy in a human body, the at least one electrode being arranged at an end region of the tube and an electrical terminal being attached to an other end region of the tube for electrically connecting the catheter to a control device. The at least one electrode and the electrical terminal are electrically conductively connected to one another by conductive tracks on a reversibly elastically extensible printed circuit board. The extensible printed circuit board forms a lateral surface of the catheter.

### CROSS-REFERENCE TO PRIOR APPLICATION

Priority is claimed to European Patent Application No. EP 19 196 488.1, filed on Sep. 10, 2019, the entire disclosure of which is hereby incorporated by reference herein.

### FIELD

The invention relates to a catheter.

### BACKGROUND

A wide variety of catheter designs are known from the prior art, which are constructed in accordance with the medical application thereof. This also includes electrode-carrying catheters, such as are described, for example, in DE 694 01 562 T2. So-called “mapping catheters” are also known, for example, for the 3-dimensional detection of the anatomy of a ventricle, which must include a high number of electrodes.

In the catheters known from the prior art, high production costs arise because the conductive tracks leading to the electrodes have to be accommodated in the catheter tube in a complex manner. It is further problematic that, due to the materials used, a limitation can result with respect to the bending radius of the catheter and, consequently, a restriction in the use thereof. Particularly in the case of cadiological applications, it is important for the catheter to tolerate narrow turns and for it to be able to be bent several times in accordance with the bending radii prescribed by the blood vessels.

### SUMMARY

In an embodiment, the present invention provides a catheter, comprising: an elongate, flexible tube comprising at least one electrode configured to carry out a diagnosis or therapy in a human body, the at least one electrode being arranged at an end region of the tube and an electrical terminal being attached to an other end region of the tube for electrically connecting the catheter to a control device, wherein the at least one electrode and the electrical terminal are electrically conductively connected to one another by conductive tracks on a reversibly elastically extensible printed circuit board, and wherein the extensible printed circuit board forms a lateral surface of the catheter.

### BRIEF DESCRIPTION OF THE DRAWINGS

The present invention will be described in even greater detail below based on the exemplary figures. The invention is not limited to the exemplary embodiments. Other features and advantages of various embodiments of the present invention will become apparent by reading the following detailed description with reference to the attached drawings which illustrate the following:

FIG. 1 shows an extensible circuit board before and after it has been formed into a tube;

FIG. 2 shows a catheter according to the invention; and

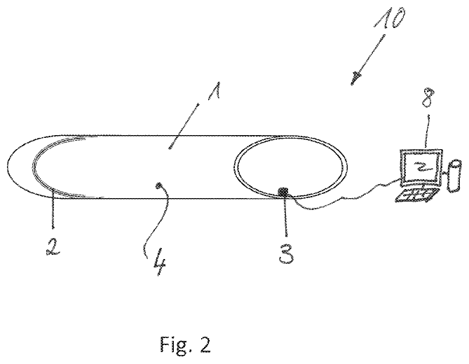

FIG. 3 shows different meander shapes of the conductive tracks.

### DETAILED DESCRIPTION

In an embodiment, the present invention provides a cost-effective and, at the same time, flexible catheter and to at least partially eliminate the disadvantages of the prior art.

In an embodiment, the present invention provides a catheter with an extensible printed circuit board having the features described herein.

According to the invention, extensible printed circuit boards have been found to be advantageous:

The catheter has the shape of an elongate, flexible tube, which could also be referred to as a hose. The catheter has at least one electrode for carrying out a diagnosis, for example a measurement such as an electrophysiological examination, or for carrying out a therapy, for example an operation such as a catheter ablation, in the human body in each case. The at least one electrode is arranged at an end region of the tube, and an electrical terminal is attached at the other end region of the tube for electrically connecting the catheter to a control device, such that a current-conducting and data-transmitting connection exists between the at least one electrode and the electrical terminal.

The at least one electrode and the electrical connection are advantageously electrically conductively connected to one another by means of conductive tracks on at least one reversibly elastically extensible printed circuit board, and the extensible printed circuit board forms the lateral surface of the catheter. Reversible extension or elastic extension signifies a reversible or non-permanent deformation.

Reversibly elastic means that the printed circuit board can extend and experiences a positive change in length (stretching) in a range of from 5-100% relative to the original, unextended length. This applies to extensions in all three spatial directions.

Such a catheter has very good flexibility and can be used for various diagnoses and therapies.

Thanks to the extensibility of the printed circuit board, it is advantageously not necessary to wind the printed circuit board spirally around the catheter tube, as is known from so-called “flexible printed circuit boards.”

It is preferred if extensions in the range of from 10 to 60% are possible.

It is particularly preferred if extensions in the range of from 15 to 35% are permitted. Extensions in this range allow sufficiently high flexibility of the catheter and catheter head with a sufficiently high number of reversible extension cycles before material fatigue occurs.

In a first variant, the printed circuit board can be arranged on the lateral surface of the tube for this purpose. To this end, the tube can be wrapped or covered by the printed circuit board, for example. It is particularly advantageous if the printed circuit board extends over the entire circumference of the lateral surface of the tube. No uneven spots then remain in the surface of the lateral surface.

In a second variant, the tube is formed by the printed circuit board. This has the advantage that the catheter can be manufactured from as few materials as possible in a few work steps.

In an advantageous further development of the catheter according to the invention, the printed circuit board is provided with conductive tracks made of copper or copper alloys. These can be applied in particular in a subtractive method. Such a catheter can be produced particularly quickly and cost-effectively. The end regions of the conductive tracks, the so-called “pads,” are not considered here to be part of the conductive tracks. The pads serving as electrons, for example, may also comprise other alloys, such as silver alloys.

It has proven to be particularly advantageous if the conductive tracks have a meander shape, in particular a rectangular, undulating, U-shaped or horseshoe-shaped meandering (meander). Meanderings (meanders) of this type are formed by concatenating rectangular, undulating, U-shaped or horseshoe-shaped basic meander shapes.

It has been shown in tests that it is particularly advantageous if the basic meander shapes of the meander shape, with their longer limbs situated between the curvatures or corners, each extend over a large portion of the circumference of the lateral surface of the tube, in particular over more than 30%, particularly preferably over 60 to 80%. The stress on the conductive tracks during bending of the tube when the catheter is used can thus be kept within a permissible range.

In a particularly advantageous and therefore preferred development of the catheter according to the invention, the printed circuit board consists of a thermoplastic polymer, for example comprising polyurethane (PU), in particular comprising a highly elastic, thermoplastic polyurethane (TPU). Such materials are particularly extensible, so that particularly flexible catheters result. In principle, other reversibly elastically extensible plastics and elastomers can also be used, e.g. ethylene propylene diene rubber, ethylene vinyl acetate, fluorine rubber, isoprene rubber, natural rubber, silicone rubber, styrene butadiene rubber or polyvinyl butyral.

The invention also relates to the use of a catheter as described above as a cardiac catheter, which is used in the diagnosis or therapy of the human heart.

The described invention and the described advantageous further embodiments of the invention constitute advantageous further embodiments of the invention also in combination with one another, insofar as this is technically reasonable.

With regard to further advantages and embodiments of the invention that are advantageous from a design and functional standpoint, reference is made to the dependent claims and to the description of exemplary embodiments, with reference to the accompanying figures.

The invention will now be explained in more detail using the accompanying figures. Corresponding elements and components are provided with the same reference symbols in the figures. For the sake of better clarity of the figures, a presentation that is true to scale has been dispensed with.

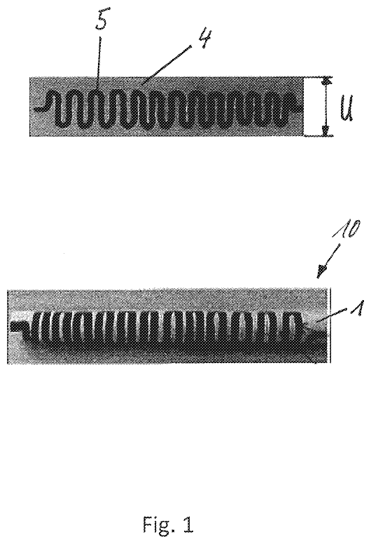

FIG. 1 shows an extensible circuit board 4 before and after it has been formed into a tube 1 of a catheter 10. On the printed circuit board 4, there are conductive tracks 5 which have a meander shape. The basic meander shapes of the conductive tracks 5 extend over a large portion of the circumference U of the lateral surface of the tube 1.

FIG. 1 shows a catheter 10 according to the invention, which has the shape of an elongate flexible tube 1. At least one electrode 2 is arranged at the one end region thereof. An electrical terminal 3 is attached to the other end region thereof. The at least one electrode 2 is connected by way of conductive tracks 5 on an elastically extensible printed circuit board 4 (not shown in greater detail here, see FIG. 1) to the electrical terminal 3 in a data-transmitting and current-conducting manner. A control device 8, which is used to control the catheter 10, is connected to the terminal 3.

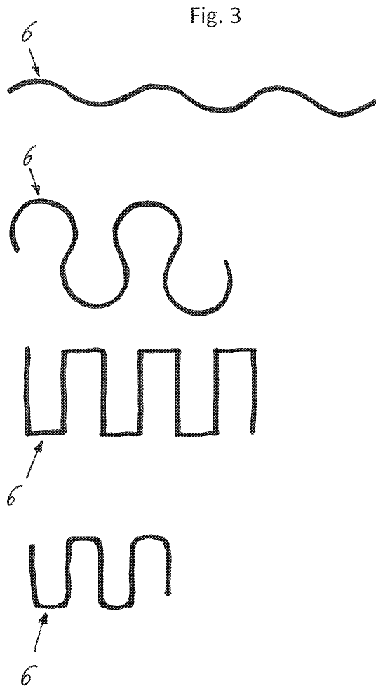

FIG. 3 shows different meander shapes which the conductive tracks 5 can have on the elastically extensible printed circuit board 4: the basic meander shape 6 can be undulating, horseshoe-shaped, rectangular or U-shaped.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

List of Reference Signs
1 Tube
2 Electrode
3 Terminal
4 Printed circuit board
5 Conductive track
6 Basic meander shape
7 —
8 Control device
9 —
10 Catheter
U Circumference of the lateral surface of the tube

What is claimed is:

1. A catheter, comprising:
an elongate, flexible tube comprising at least one electrode configured to carry out a diagnosis or therapy in a human body, the at least one electrode being arranged at an end region of the tube and an electrical terminal being attached to an other end region of the tube for electrically connecting the catheter to a control device,
wherein the at least one electrode and the electrical terminal are electrically conductively connected to one another by conductive tracks on a reversibly elastically extensible printed circuit board, and
wherein the extensible printed circuit board forms a lateral surface of the catheter.

2. The catheter according to claim 1, wherein the extensible printed circuit board is arranged on the lateral surface of the tube.

3. The catheter according to claim 2, wherein the extensible printed circuit board extends over an entire circumference of the lateral surface of the tube.

4. The catheter according to claim 1, wherein the extensible printed circuit board forms the tube.

5. The catheter according to claim 1, wherein the extensible printed circuit board comprises conductive tracks comprising copper or copper alloys.

6. The catheter according to claim 1, wherein the conductive tracks have a meander shape comprising a rectangular, undulating, U-shaped, or horseshoe-shaped meandering.

7. The catheter according to claim 6, wherein the meander shape comprises longer limbs, the longer limbs being situated between curvatures or corners of the meander shape, each of the longer limbs extending over a large portion of a circumference of the lateral surface of the tube, the large portion of the circumference comprises more than 30%.

8. The catheter according to claim 1, wherein the extensible printed circuit board comprises a thermoplastic polymer.

9. The catheter according to claim 7, wherein the large portion of the circumference comprises 60 to 80%.

10. The catheter according to claim 8, wherein the thermoplastic polymer comprises polyurethane.

11. The catheter according to claim 10, wherein the polyurethane comprises a highly elastic, thermoplastic polyurethane.
